(19) United States
(12) Patent Application Publication
(10) Pub. No.: US 2024/0233932 A1
(43) Pub. Date: Jul. 11, 2024

(54) USING PREDICATE DEVICE NETWORKS TO PREDICT MEDICAL DEVICE RECALLS

(71) Applicant: Regents of the University of Minnesota, Minneapolis, MN (US)

(72) Inventors: Soumya Sen, Minneapolis, MN (US); Yi Zhu, Minneapolis, MN (US); Pinar Karaca Mandic, Minneapolis, MN (US)

(21) Appl. No.: 18/408,061

(22) Filed: Jan. 9, 2024

Related U.S. Application Data
(60) Provisional application No. 63/479,339, filed on Jan. 10, 2023.

(51) Int. Cl. G16H 40/40

(52) U.S. Cl. CPC G16H 40/40

(57) ABSTRACT

A system for estimating a recall probability for a medical device includes a predicate device database having stored thereon relationships between a plurality of medical devices. A processor is in communication with the predicate device database and is configured to generate a network of medical devices having a relationship to a focal medical device using the predicate device database. The generated network is used to form features, which are applied to a predictive model to determine the recall probability.

### CROSS-REFERENCE TO RELATED APPLICATIONS

The present application is based on and claims the benefit of U.S. provisional patent application Ser. No. 63/479,339, filed Jan. 10, 2022, the content of which is hereby incorporated by reference in its entirety.

### SUMMARY

A system for estimating a recall probability for a medical device includes a predicate device database having stored thereon relationships between a plurality of medical devices. A processor is in communication with the predicate device database and is configured to generate a network of medical devices having a relationship to a focal medical device using the predicate device database. The generated network is used to form features, which are applied to a predictive model to determine the recall probability.

In accordance with a further embodiment, a method for estimating a recall probability for a focal medical device includes generating a predicate device network for a focal medical device using a computer system and using the predicate device network to generate features for the focal medical device using a computer system. The features are applied to a predictive model with the computer system, wherein the predictive model has been trained on training data to estimate a medical device recall probability from features associated with a predicate device network. The probability that the focal medical device will be recalled within a time window generated by the predictive model is output.

In accordance with a still further embodiment, a method includes applying features to a multi-hop Graph Convolution Network having an adjacency matrix that is determined from a predicate device network for a focal medical device and using the output of the Graph Convolution Network to determine a probability of the focal medical device being recalled.

The foregoing and other aspects and advantages of the present disclosure will appear from the following description. In the description, reference is made to the accompanying drawings that form a part hereof, and in which there is shown by way of illustration one or more embodiments. These embodiments do not necessarily represent the full scope of the invention, however, and reference is therefore made to the claims and herein for interpreting the scope of the invention.

### BRIEF DESCRIPTION OF THE DRAWINGS

FIG. 1 illustrates an example medical device recall predicting system and its associated components, according to some embodiments described in the present disclosure.

FIG. 2 is a flowchart setting forth the steps of an example method for extracting medical device data from an unstructured data source retrieved from a regulatory authority.

FIG. 3 shows the conversion of a predicate device network to a 1-hop network.

FIG. 4 shows the conversion of a predicate device network to a 2-hop network.

FIG. 5 shows the conversion of a predicate device network to a 3-hop network.

FIG. 6 provides a block diagram of a predictive model in accordance with one embodiment.

FIG. 7 provides a flow diagram of a method of training a predictive model.

FIG. 8 is a block diagram of example components that can implement a system in accordance with one embodiment.

FIG. 9 provides an initial user interface.

FIG. 10 provides an example of a basic information user interface.

FIG. 11 provides an example of a predicate devices user interface.

FIG. 12 provides an example of a recall predictions user interface.

FIG. 13 provides an example of a recall probability comparison user interface.

### DETAILED DESCRIPTION

Described herein are systems and methods for predicting medical device recalls using machine learning. Multiple data sources are leveraged, and machine learning algorithms are developed to provide an artificial intelligence/machine learning (“AI/ML”)-based decision support system to analyze medical device histories and predict their recalls.

Medical device adverse events, such as recalls, are important to patient safety. Studies show that hundreds of devices are recalled annually under Class I and Class II categories. Class I occurs when devices may cause “serious health problems or death,” while Class II occurs when devices may cause “temporary or reversible” health problems or when there is a “slight chance” that they “will cause serious health problems or death.” Accurately and timely predicting medical device recalls is important for preventing medical malpractices, which greatly threaten the lives of patients and the reliability of healthcare systems.

The systems and methods described in the present disclosure address the unmet need for an efficient end-to-end system or method to predict medical device recalls based on quantitative machine learning and big data technologies. One of the main reasons these technologies have yet to be widely used is that publicly available data for recall predictors is lacking or not in an analysis-ready format to be directly applied for building predictive models. Inspired by the long-lasting concerns of FDA's 510(k) medical device clearance pathway (devices approved mainly based on similarity to predicate devices rather than demonstrated efficacy), we develop a machine learning framework for predicting medical device recalls based on the predicate device and device recall information embedded in publicly available 510(k) documents and device recall public records.

The systems and methods described in the present disclosure provide several advantages over existing systems for monitoring medical device recalls. As one example, the disclosed systems and methods provide analytical insights within predicate device and device recall databases that are created from data extracted from regulatory authority data sources. For instance, the predicted probabilities of medical device recalls across different time windows (e.g., probability of recalls in 2-year or 3-year time windows) can be estimated. This insight is advantageous for medical device manufacturers and regulatory bodies as they consider marketing new medical devices through the 510(k) clearance pathway, or monitoring the chances of recalls for existing medical devices. As another example, the disclosed systems and methods create a predicate network for each medical device (including its predecessors and successors) that not only presents the citing relationship among devices but also shows which devices are recalled or have a high recall probability. The shortest citing path (in terms of network hops and/or approval year gap) from the focal device to the recalled device can also be computed and visualized to help evaluate the recall probability of the focal device. As yet another example, the disclosed systems and methods provide database creation and visualization processes that are automatic without requiring manual data scraping from data sources websites, making the analysis and visualization tasks scalable.

In one aspect, the disclosed systems and methods implement an automated data extraction to extract medical device data from regulatory authority data sources and/or other suitable data sources. For example, an algorithmic technique for natural language processing (“NLP”) is used to automatically extract analysis-ready predicate device and device history information embedded in public 510(k) documents from FDA and recall records. The NLP technique is used to extract predicate and recall information from unstructured public 510(k) documents and recall records. Two databases can be constructed with the extracted information (i.e., a predicate device database and a device recall database), and can be complemented with additional data collected from medical device manufacturers or other data sources.

It is another aspect of the present disclosure to provide systems and methods for creating and visualizing a predicate device network. The creation of a predicate device network that captures the interrelationships across devices improves the ability to visualize the interrelationships between a focal medical device and its predecessors and successors. Key network features can then be readily extracted from the created predicate device network and connected predicate device database.

Another advantage of the systems and methods described in the present disclosure is their ability to integrate data from multiple different data sources. For instance, the systems and methods are capable of integrating across multiple created databases and public data sources, such as the created predicate device and device recall databases and the public device clearance data. Machine learning algorithms, models, or programs can then be developed and utilized to predict medical device recalls for a focal medical device, leveraging the data spanned across the predicate device network.

A user interface is also provided for demonstrating, visualizing, otherwise displaying the data insights and the predicted recall probabilities of different devices over time horizons to facilitate exploratory analytics.

The systems and methods described in the present disclosure can be useful for a number of applications. As one example, the systems and methods can be advantageous to medical device manufacturers. For instance, the disclosed systems and methods can help manufacturers search and visualize devices that are similar to a new medical device (or related technologies) in order to identify ideal candidates to cite as predicate devices. Second, the analysis insights of medical device recalls can help the manufacturer understand the recall patterns of existing devices, which can help the manufacturer design safer devices that actively avoid depending on recalled devices or devices with high recall probabilities as predicate devices. The manufacturer can also monitor the recall probabilities of their own devices across time. By monitoring such information, manufacturers can take early actions to those devices with high recall probabilities (e.g., special checks or replacements) to avoid the eventual recalls that could bring unexpected financial loss.

An online interactive visualization tool can be provided based on these systems and methods to fully demonstrate the created databases, predicate device network, and predicted device recall probabilities. A mobile version of the visualization tool can also be built to support mobile access to useful medical device information.

With the analysis insights of the device recall probabilities, insights can be provided to device manufacturers to help them evaluate which of their devices are likely to be recalled and at what time. This service can largely reduce the recall problem faced by manufacturers, which can threaten their financial stability and company reputation. A warning notification of devices with high recall risks based on the analysis insight provided by the disclosed systems and methods can also be sent to manufacturers to help them take early actions before actual recalls. More broadly, the data can be useful to any stakeholders (e.g., regulators, lawyers) interested in analyzing any medical device's history, related devices, and their safety data.

### System Overview

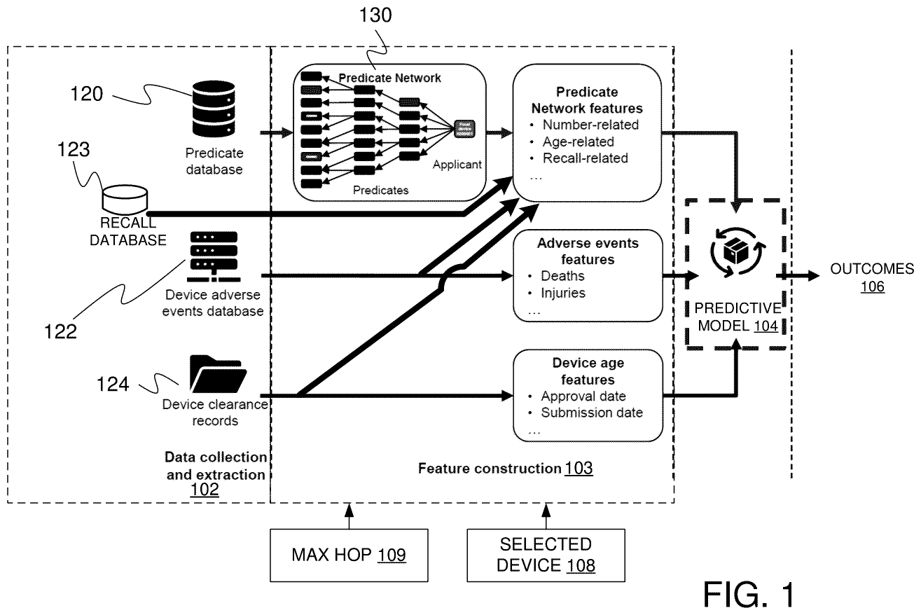

FIG. 1 depicts an overview of the disclosed systems and methods for predicting medical device recall probabilities described in the present disclosure. The medical device recall prediction system 100 is an end-to-end predictive system that includes data collection and extraction 102, feature construction 103, predictive models 104, and predictions 106.

### Data Collection and Extraction

Data collection and extraction 102 includes steps and components for constructing and/or updating a predicate device database 120, constructing and/or updating a device adverse events database 122, constructing and/or updating a recall database 123, constructing and/or updating one or more device clearance records 124 that may contain additional medical device features retrieved from other data sources. Adverse events database 122 includes adverse events such as deaths and injuries associated with all 510(k) devices including the number of injuries, the number of deaths and the number of malfunctions associated with each device. Recall database 123 contains the recall history (e.g. recall date, resolving date, recall type, current recall status) for each device that has been recalled. Device clearance records 124 include basic device information such as device approval date, manufacturer, product code, medical specialty of all 510(k) devices.

Predicate device database 120 provides relationships between devices that together provide a graph structure with devices as nodes and predicate relationships between devices as edges. In accordance with one embodiment, each predicate relationship is directional such that a device on one end of predicate relationship is identified as a predecessor and the device on the other end of the predicate relationship is identified as the successor with the predecessor being identified as a predicate device of the successor. Each device node can be associated with zero, one or multiple edges in which the device is a successor and with zero, one or multiple edges in which the device is a predecessor.

Device adverse events database 122 is loaded with data retrieved from the Manufacturer and User Facility Device Experience (MAUDE) database, which records device adverse events (e.g., deaths, injuries, etc.) reports. Device recall database 123 is loaded with data retrieved from an FDA device recall database. Device clearance records 124 is loaded with data retrieved from FDA clearance records, which contain the basic information (e.g., device approval date, manufacturer, product code, etc.) of all 510(k) devices.

In accordance with one embodiment, constructing predicate device database 120 involves extracting information from publicly available FDA 510(k) regulatory submission text files, which each directly identifies the predicates for a respective medical device. In accordance with one embodiment, a Natural Language Processing (NLP) technique is used to automatically extract predicate device information from these documents.

There are a few challenges to this task. First, the 510(k) documents do not follow a standard template, and the texts that indicate the information on predicate devices are not structured, which makes it challenging to locate and extract the content of interest from these documents. One efficient way to solve this problem is to locate the desired information based on some “rule-based” NLP techniques. For example, predicate device information often follows behind some locators (or keywords) such as “predicate”, “equivalent”, “reference”, and their derived forms.

As another challenge, the predicate device information identifiers in the 510(k) documents are not expressed in a single format. The majority of the documents cite their predicate devices by K number, a unique identification number associated with a 510(k) file that starts with the letter “K” and followed by six digits, such as K193645 and K033669. The first two digits of the K number indicate the 510(k) receiving year by the FDA of the focal device, and the remaining four digits are an identification code. Some 510(k) documents, however, only cite the name of the predicate device without a K number, while others may not cite any predicate device.

As yet another challenge, each 510(k) document may cite more than one predicate device. Therefore, all of the “K-number-like” strings after the locators must be extracted. There could also be exceptions that the K number is not cited after the locators, but are cited in other parts of the document, so a search for “K-number-like” strings in the entire document is needed to avoid missing predicate devices.

Another challenge with extracting medical device data from 510(k) data sources is that, in cases where 510(k) cites multiple predicates, the relative importance of each predicate device can be unclear. Usually, the first K number cited after the locators is the “primary” predicate device, whereas those devices that follow behind, and those cited in the other parts of the document are considered to be less relevant. To account for this, the returned lists of predicate devices can be sorted in the correct order with the primary predicate device at the very front. Furthermore, some documents explicitly indicate that a certain predicate device is the “primary predicate”. Incorporating methods like “n-gram” to identify two-word or three-word strings similar to the locator string “primary device” can be used to locate the primary predicate devices that usually follow right behind the locator.

As still another challenge, not all 510(k) documents are in a machine-readable text format, as some of them are in an image format or other encoding, which requires conversion to standard text format to be readable.

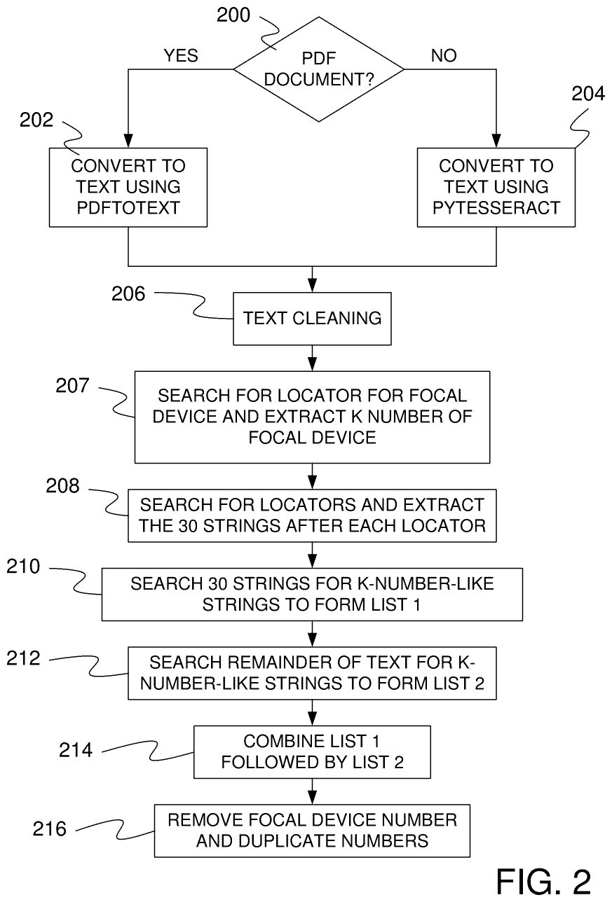

FIG. 2 provides a flow diagram of a method in accordance with one embodiment for extracting a list of predicate devices from a 510(k) document. In step 200, the format of the document is examined to determine if the document is in PDF format or some other format. If the document is in the PDF format, the document is applied to a PDF-to-text converter at step 202. In accordance with one embodiment, the PDF-to-text converter is the “pdftotext” package in python. If the document is in another format, a different converter is used at step 204 to convert the document into text. For example, the “pytesseract” package in python can be used to read the documents in other formats at step 204.

At step 206, standard text cleaning is applied to the text produced at steps 202 and 204.

At step 207, a set of “locators” (i.e., keywords that are followed a K number) are searched for in the cleaned text to determine the K number of the focal device of this 510(k) document. As a non-limiting example, the following keywords can be defined as locators: “510(k) Number” and “510(k) #”. When a keyword is located, the string beginning with “K” after the keyword is extracted and is used as the K number of the focal device.

At step 208, a set of “locators” (i.e., keywords that are followed by one or more K numbers) are searched for in the cleaned text. As a non-limiting example, the following keywords can be defined as locators: “predicate”, “predicates”, “predicated”, “equivalent”, “equivalence”, “equivalency”, “equivalented”, “equivalences”, “reference”, “references”, “referenced”, “primary predicate”. When a keyword is located, a preset number of strings (e.g., the first 30 strings) right after each identified locator is extracted from the text.

In step 210, the extracted strings are searched for “K-number-like” strings. These K-number-like strings are then sorted by the order in which they appear in the document from which they were extracted, as the order of predicate devices is important. These K numbers form the first data list.

To account for the possibility that some K numbers may be scattered in other parts of the document, the strings left in the document after extracting the locator-following strings are searched at step 212 for other “K-number-like” strings and any identified strings are returned to avoid any omission. The K numbers returned in this step form the second data list.

At step 214, the first and second data lists are combined to form an initial predicate device list (“PDL”). The PDL is then searched at step 216 to remove the K number of the focal device itself if present and to remove any duplicate predicate numbers. In addition, clearance records 124 are searched to obtain the clearance record for each K number in the PDL. If a clearance record cannot be found for the K number or the approval for the K number occurred after the approval of the focal device, the K number is removed from the predicate device list. The resulting list forms the final Predictive Device List (PDL).

The predicate device list is then used to update predicate database 120. Specifically, a record for the focal device of the document is created in predicate database 120 if it is not already present. One of the predicate devices in the predicate device list is then selected. A search of predicate database 120 is then performed for the device. If the device is not present in predicate database 120, the device is added to predicate database 120. A relationship is then added between the focal device and the selected predicate device with the predicate device designated as the predecessor device and the focal device designated as the successor device. These steps are repeated for each device in the predicate device list.

Given the complex nature of 510(k) documents in terms of format, component, structure, etc., this framework is specially designed to address issues in 510(k) documents, though it has the potential to be generalized to account for similar situations for extracting information of interest when the information follows a regular rule and there exist certain “locators” to help locate the information.

### Feature Construction

Feature construction 103 uses a selected device 108, referred to as the focal device, and predicate database 120, device recall database 123, device adverse events database 122 and device clearance records 124 to construct a set of features that can be applied to the predictive model. These features are divided into predicate features that are derived from attributes of predicate devices of a device and stand alone features that are derived only from attributes of the device. Further, some of the features are static features (time-invariant in that they do not change from year to year, while other features are time-varying. Time-varying features are computed by year.

To form the predicate features, a predicate network 130 is formed for selected device 108 using predicate database 120. In predicate network 130, devices that are connected directly to selected device 108 are said to be one hop from selected device 108, devices that are connected to selected device 108 through one intermediary device are said to be two hops from selected device 108 and devices that are connected to device 108 through two intermediary devices are said to be three hops from selected device 108. If there are two paths between a device and selected device 108, the shortest path is used.

Predicate features and device features are then determined for selected device 108. For many of the predicate features, the feature is dependent on the number of hops from selected device 108 to a predicate device. For example, there is one feature for the number of recalled devices that are one hop from selected device 108, a second feature for the number of recalled devices that are two or less hops from selected device 108 and a third feature for the number of recalled devices that are three or less hops from selected device 108. A summary of the features that are constructed is reported in Table 1, with the number of features that are created shown in parentheses.

TABLE 1
Constructed Features

| | |
|---|---|
| Feature category | Feature description |

Predicate network features (92)
(in one-hop, one + two-hop, one + two + three-hop networks)

| | |
|---|---|
| Number-related features | Number of predicates (3). |
| (static) | Single-predicate existence indicator (only in one-hop network) (1). |
| Age-related features | Max, min, std, mean, and median of the approval decision date difference |
| (static) | between predicate devices and applicant devices (15). |
| | Ten-year predicate existence indicator (only in one-hop network) (1). |
| Recall-related features | Number and percentage of recalls among predicates (6). |
| (temporal) | Number and percentage of ongoing recalls among predicates (6). |
| Adverse-events-related | Number of death/injury/malfunction/other event reports among predicates |
| features | (12). |
| (temporal) | Percentage of predicates with at least one death/injury/malfunction/other |
| | event report (12). |
| Other statistics | Unique number and entropy of product codes/medical specialties/applicant |
| (temporal) | companies among the predicates (18). |
| | Number and percentage of predicates with a different product code/medical |
| | specialties/applicant companies than focal device (18). |

Device standalone features (7)

| | |
|---|---|
| Adverse events features | The number of death, injury, malfunction, and other event reports (4). |
| (temporal) | |
| Date features | Date difference between current date and approval decision date (time- |
| (temporal/static) | varying) (1). |
| | Date difference between approval decision date and 510(k) submission |
| | receiving date (time-invariant) (1). |
| | Date difference between approval decision date and Jan. 1, 2003, the first |
| | date of the study period (time-invariant) (1). |

The features above are also determined for each predicate device that is within a maximum number of hops 109 from selected device 108. Thus, if the maximum number of hops 109 is three, each device that is within three hops of selected device 108 will have these features constructed for it. In accordance with some embodiments, an additional feature indicating whether a device has been recalled is added to each predicate device within the maximum number of hops of selected device 108.

### Predictive Model

In accordance with one embodiment, predictive models 104 are designed to utilize the characteristics of the device predicates such as how many of the predict devices have been recalled as well as the timing of recalls to predict the likelihood that a selected device will be recalled in a set of time windows. Instead of creating a single model, a set of models is created for each year. Within each set of models, there is a separate model for each of the set of time windows such as a one-year window, a two-year window, a three-year window, a four-year window and a five-year window.

In accordance with one embodiment, a multi-hop Graph Convolution Network is used to model the impact that a recalled device in a predicate network has on the probability of a focal device being recalled. Traditional GCNs learn a network node's representation by aggregating the features of neighbors and the node itself with learned parameters. To incorporate the characteristics of neighbors connected several hops away, GCNs add multiple layers or uses larger convolution filters in unstructured shapes. Like other deep learning techniques, GCN uses an optimization function (e.g., Adam) and backpropagation process to optimize the filter weights used in each layer. Specifically, a GCN layer is defined as:

$${H}^{l} = σ ( {\tilde{D}}^{- \frac{1}{2}} \tilde{A} {\tilde{D}}^{- \frac{1}{2}} {H}^{l - 1} {W}^{l - 1} )$$

where H^{l} and H^{l-1} are the representation of a node in the current and previous layers, Ã is the node's adjacency matrix with self-connection, \tilde{D}^{-1/2}Ã\tilde{D}^{-1/2} is the degree-normalized adjacency matrix with self-connection, W^{l-1} is a matrix of filter weights learned in the previous layer, and σ is an activation function.

Traditional GCNs have two main limitations. First, the deeper networks or larger filters introduce an over-smoothing problem, as too many nodes included in feature aggregation make all nodes' features similar. Second, models with multiple layers introduce redundant information, as nodes connected via multiple hops are computed repeatedly in each layer, resulting in suboptimal node representations. These issues are salient in the medical device predicate network as device recalls may be influenced by other devices connected several hops away and two devices can be connected not only via one hop. To address these issues, we use a multi-hop GCN to learn device representations. Instead of computing from the one-hop adjacency matrix in each stacked GCN layer, we pre-construct the adjacency matrices in different network hops separately, allowing each node to directly connect with distant neighbors.

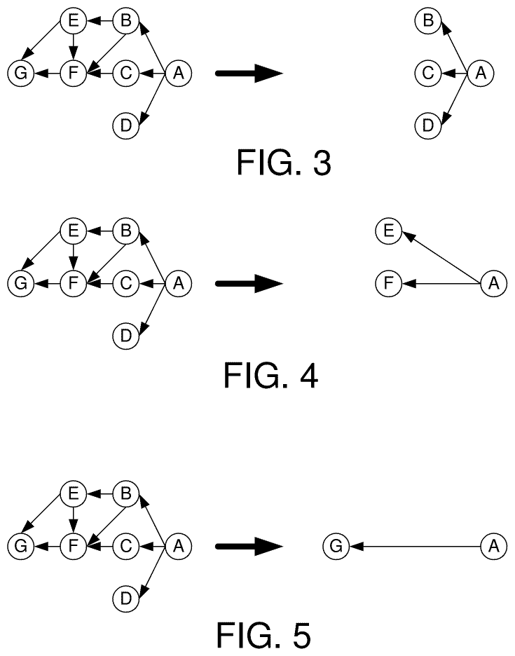

FIGS. 3, 4 and 5 show the conversion from a complete predicate network 300 to 1-hop, 2-hop and 3-hop networks for a device A. In FIG. 3, devices B, C, and D are shown to be part of A's 1-hop network while devices E, F and G are not. In FIG. 4, devices E and F are part of A's 2-hop network but devices B, C, D and G are not. In FIG. 5, device G is part of A's 3-hop network but devices B, C, D, E and F are not. Note that even though device F can be reached through 3 hops (A-B-E-F), it can also be reached through 2 hops (A-C-F). When a device can be reached through different numbers of hops, the device is included in the network with the least number of hops.

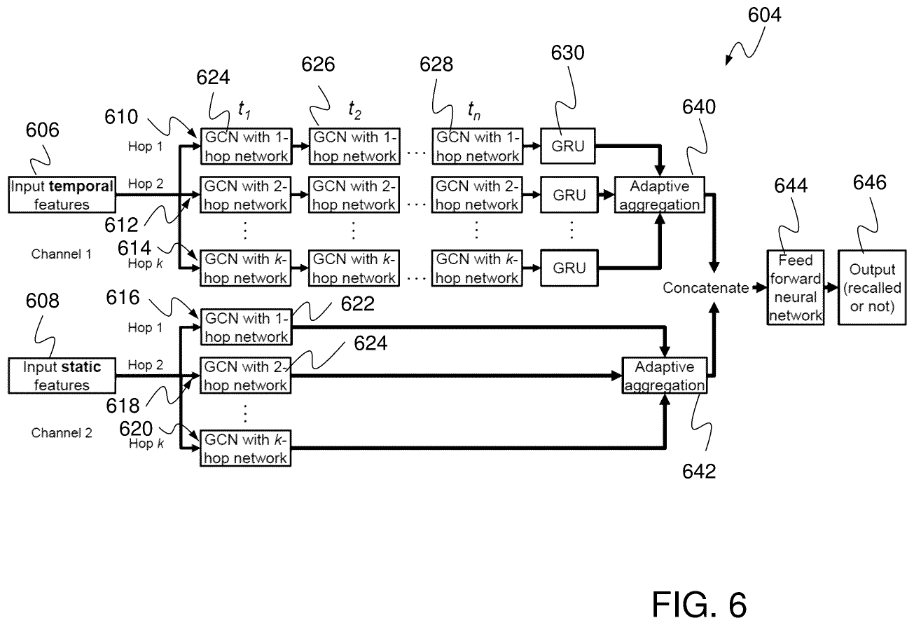

FIG. 6 provides a block diagram of a predictive model 604, which is one of the predictive models 104 in accordance with one embodiment. Predictive model 604 is referred to as DeepPredicate. In predictive model 604, the input features are divided into temporal features 606, which are able to change from year to year, and static feature 608, which are time invariant. Temporal features 606 are applied to multiple branches, each associated with a different hop network. For example, branch 610 is associated with a 1-hop network, branch 612 is associated with a 2-hop network, and branch 614 is associated with a k-hop network. Similarly, static features 608 are applied to multiple branches 616, 618 and 620, each associated with a different hop network.

Within each of branches 616, 618 and 620, static features 608 are applied as input to a respective Graph Convolution Network with a respective adjacency matrix that is dependent on the hop network associated with branch. For example, in branch 616, the adjacency matrix in GCN 622 is based on the 1-hop network and in branch 618, the adjacency matrix in GCN 624 is based on the 2-hop network. Each branch's GCN produces a separate representation for the focal device based on a different predicate device network topology resulting in k parallel representations of the focal device from static features 608.

To account for the temporal variations of predicate network features (e.g., the number of recalled predicates across time), branches 610, 612 and 614 for the temporal features include a separate layer of the GCN for each of a sequence of years t₁, t₂ . . . tₙ. In particular, the value of the temporal features for the corresponding year are input to the GCN layer for that year as well as the representation of the focal device and the weights from the previous GCN layer. For example, GCN layer 626 in branch 610 receives the temporal features for year t₂ and the representation of the focal device from GCN layer 624 of year t₁ and GCN layer 626 provides a representation of the focal device to the next GCN layer. In addition, the representation of the focal device produced by each GCN layer in a branch is provided to a sequence processing model such as a Gated Recurrent Unit (GRU) for the branch. For example, GRU 630 receives the representations of the focal device produced by GCN layers 624, 626 and 628 in branch 610.

The GRU is trained to utilize GCN outputs at valuable time points and to discard GCN outputs at less valuable time points. LSTM and transformer models are feasible alternatives to GRU within our method.

The branches for the temporal features and the branches for the static features are combined using respective adaptive aggregation 640 and 642. Adaptive aggregation 640 aggregates the outputs of the branches' GRUs while adaptive aggregation 642 aggregates the outputs of the branches' GCN.

The adaptive aggregations are performed based on two considerations. The first is the different importance weights of the branch-specified node representations. Predicate devices in each branch are connected to the focal device through different numbers of network hops. Depending on the predicate devices' connection closeness (connecting via fewer hops means closer) to the focal device, the computed node representations (a node here is an focal device) may have different importance weights. The second consideration is the temporal dependencies of the node representations across branches. Predicate devices are ordered chronologically across branches, where the higher-ordered branches include older predicate devices, and lower-ordered branches include newer predicate devices. Hence, device characteristics may depend on each other sequentially across branches.

Our adaptive aggregation function uses an attention-based GRU model to account for the different importance of each branch and to learn the temporal dependencies across branches. Specifically, the attention mechanism is used to capture the different importance of each branch, and the GRU is applied to learn the temporal dependencies across branches.

The adaptive aggregation is applied to temporal feature and static feature channels separately and the outputs of the aggregation layers are concatenated before connecting to a fully connected feed-forward neural network 644 and, during testing, activating the prediction 646. Specifically, feed-forward neural network 644 provides a probability of a recall and activation function 646 selects either “recall” or “no recall” based on the probability. Activation function 646 allows the output of prediction model 104 to be compared to what actually happened for a test device.

In accordance with one embodiment, a set of predictive models 604 is created for each year. Within the set of predictive models 604 is a separate predictive model 604 for each of a set of time windows, including one year, two year, three year, four year, and five year. The predictive model 604 for a time window of x years is trained on temporal features that end x years before a current year and the recall status of the focal device during the current year is used as the target value for the model. Thus, each predictive model 604 provides a probability that the focal device will be recalled within the time window associated with the predictive model.

Compared to traditional T-GCN, the DeepPredicate more efficiently captures the network structure by learning the node representations directly from distant connected nodes, reducing over-smoothing and duplicated computation. It can also simultaneously learn the temporal patterns of node representations across temporal input and network hops, which are both important for improving medical device recall prediction.

### Predictive Algorithm/Model Training

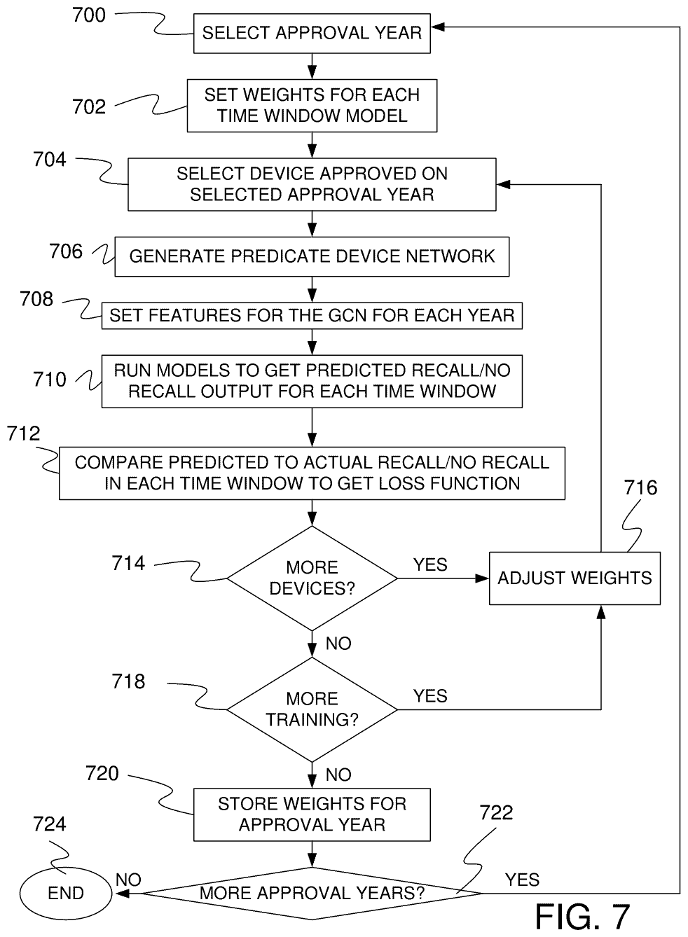

Referring now to FIG. 7, a flowchart is illustrated as setting forth the steps of an example method for training predictive models 104.

In step 700, an approval year is selected. At step 702, initial weights for the various neural networks found in each time window's predictive model 604 are set. At step 704, a device that was approved during the selected approval year is selected from a collection of training data. At step 706, a predicate device network is formed for the selected device using the training data. At step 708, the predicate device network is used to generate a set of features for each of the years spanned by the GCN's beginning with the approval year. These are the same features as discussed above.

At step 710, each time window's model is run using the training features to produce a prediction of recall/no recall for the selected device during the model's time window. At step 712 the recall/no recall prediction is compared to the actual recall/no recall status of the selected device during each respective time window to produce a loss function value for each time window's model.

At step 714, the training method determines if there are more devices that were approved during the approval year. If there are more devices, the weights of the models are adjusted at step 716 based on the loss function. For instance, the model weights can be updated in order to minimize the loss according to the loss function. Different types of training processes can be used to adjust the bias values and the weights of the models such as gradient descent, Newton's method, conjugate gradient, quasi-Newton, Levenberg-Marquardt, among others.

The process then returns to step 704 to select another device that was approved during the selected approval year. Steps 704, 706, 708, 710, 712, 714 and 716 are repeated until all of the devices approved during the selected approval year have been applied to the models.

After all of the devices have been applied at step 714, the method determines if a training condition has been met at step 718. The training condition may correspond to, for example, a predetermined number of training examples being used, a minimum accuracy threshold being reached during training and validation, a predetermined number of validation iterations being completed, and the like.

When the training condition has not been met, the weights are once again adjusted at step 716 and the training returns to step 704 to pass through the list of devices approved that year again. Steps 704, 706, 708, 710, 712, 714 and 716 are repeated until all of the devices approved during the selected approval year have been applied to the models another time.

When the training condition has been met at step 718 (e.g., by determining whether an error threshold or other stopping criterion has been satisfied), the current model weights represent the trained time window models for the selected approval year. As such, these weights are stored at step 720 as the time window models for the selected approval year.

At step 722, the training process determines if there are more approval years. If there are more approval years in the training data, the process returns to step 700 to select the next approval year. When all of the approval years in the training data have been used for training, the training process ends at step 724.

The predictive models can be constructed or otherwise trained based on training data using one or more different learning techniques, such as supervised learning, unsupervised learning, reinforcement learning, ensemble learning, active learning, transfer learning, or other suitable learning techniques for neural networks.

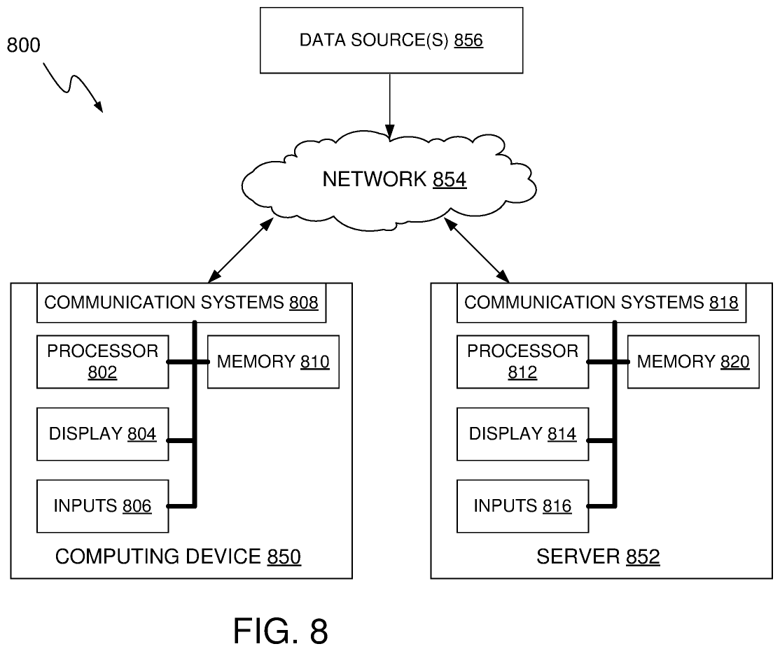

Referring now to FIG. 8, an example of hardware 800 that can be used to obtain recall information about a device, include a recall probability for a device, in accordance with one embodiment.

In FIG. 8, a processor 812 in a server 852 downloads data used to create predicate database 120, recall database 123, device adverse events database 122 and device clearance records 124 from one or more data source servers 856 through a network 854 using a communication system 818. Server 852 stores the downloaded data in a memory 820. Memory 820 also contains the weights that describe the time window predictive models for each approval year. These weights may be determined during training executed on server 852 or may be loaded from another computer where the training above took place.

Processor 812 generates user interfaces that allow users to request recall information about a selected device and to return that information to the user. In particular, processor 812 sends user interfaces through communications system 818 to a communications system 808 on a user computing device 850. A processor 802 on computing device 850 displays the user interfaces on a display 804 and receives inputs from the user through input devices 806. Computing device 850 includes a memory 810 that stores computer instructions that allow computing device 850 to display the user interfaces, receive inputs from the user and communicate with server 852.

Server 852 may optionally include a display 814 and inputs 816 to allow engineers to interact with server 852 directly.

Processors 802 and 812 can be any suitable hardware processor or combination of processors, such as a central processing unit (“CPU”), a graphics processing unit (“GPU”), and so on. In some embodiments, display 804 can include any suitable display devices, such as a liquid crystal display (“LCD”) screen, a light-emitting diode (“LED”) display, an organic LED (“OLED”) display, an electrophoretic display (e.g., an “e-ink” display), a computer monitor, a touchscreen, a television, and so on. In some embodiments, inputs 806 can include any suitable input devices and/or sensors that can be used to receive user input, such as a keyboard, a mouse, a touchscreen, a microphone, and so on.

In some embodiments, communications systems 808 and 818 can include any suitable hardware, firmware, and/or software for communicating information over communication network 854 and/or any other suitable communication networks. For example, communications systems 808 and 818 can include one or more transceivers, one or more communication chips and/or chip sets, and so on. In a more particular example, communications systems 808 can include hardware, firmware, and/or software that can be used to establish a Wi-Fi connection, a Bluetooth connection, a cellular connection, an Ethernet connection, and so on.

In some embodiments, memories 810 and 820 can include any suitable storage device or devices that can be used to store instructions, values, data, or the like, that can be used, for example, by processors 802 and 812 to present content using displays 804 or 814, to communicate with server 852 or computing device 850 via communications system(s) 808 and 818, and so on.

Memories 810 and 820 can include any suitable volatile memory, non-volatile memory, storage, or any suitable combination thereof. For example, memories 810 and 820 can include random-access memory (“RAM”), read-only memory (“ROM”), electrically programmable ROM (“EPROM”), electrically erasable ROM (“EEPROM”), other forms of volatile memory, other forms of non-volatile memory, one or more forms of semi-volatile memory, one or more flash drives, one or more hard disks, one or more solid state drives, one or more optical drives, and so on. In some embodiments, memories 810 and 820 can have encoded thereon, or otherwise stored therein, a computer program for controlling operation of computing device 850 and server 852, respectively. In such embodiments, processors 802 and 812 can respectively execute at least a portion of the computer program to present content (e.g., images, user interfaces, graphics, tables), receive content from communication network 854, transmit information to communication network 854, and so on. For example, processor 812 and the memory 820 can be configured to receive a device identifier from computing device 850, apply the received device identifier as selected device 108 to feature construction 103 and apply the constructed features to the predictive models to obtain probabilities that the selected device will be recalled in a set of time windows as discussed above.

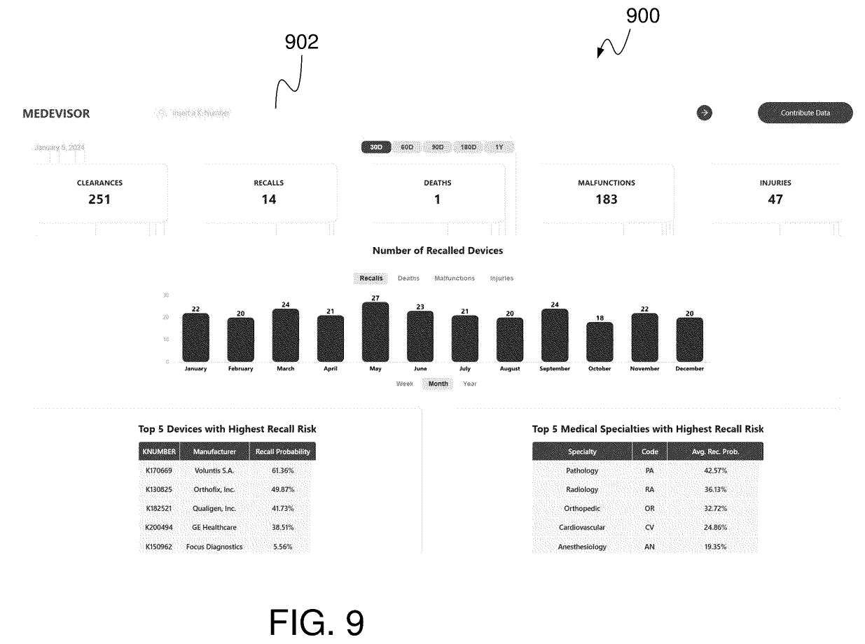

Examples of the user interfaces generated by server 852 and displayed on display 804 of computing device 850 are shown in FIGS. 9-13. FIG. 9 provides an initial user interface 900 that general information about recalls across a number of different devices. User interface 900 includes a text input box 902 that receives a K number for a device that is to be used as the focal device.

After a K number is submitted through input box 902, server 852 receives the number from computing device 850 and uses the number to generate features as discussed above. The features are applied to the models to generate probabilities of the focal device with the submitted K number being recalled during different time windows.

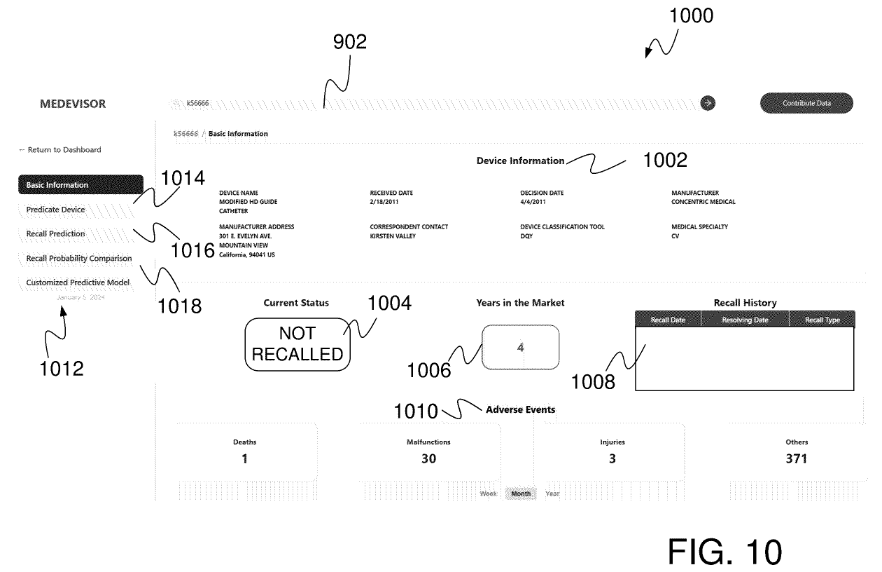

Server 852 then returns basic information user interface 1000 of FIG. 10, which includes device information 1002 for the selected device, current recall status 1004 of the selected device, years on the market 1006 of the selected device, recall history 1008 of the selected device adverse events 1010 of the selected device and menu 1012. Menu 1012 includes selection tab 1014 for requesting a predicate devices page, selection tab 1016 for requesting a recall prediction page, and selection tab 1018 for requesting a recall probability comparison page.

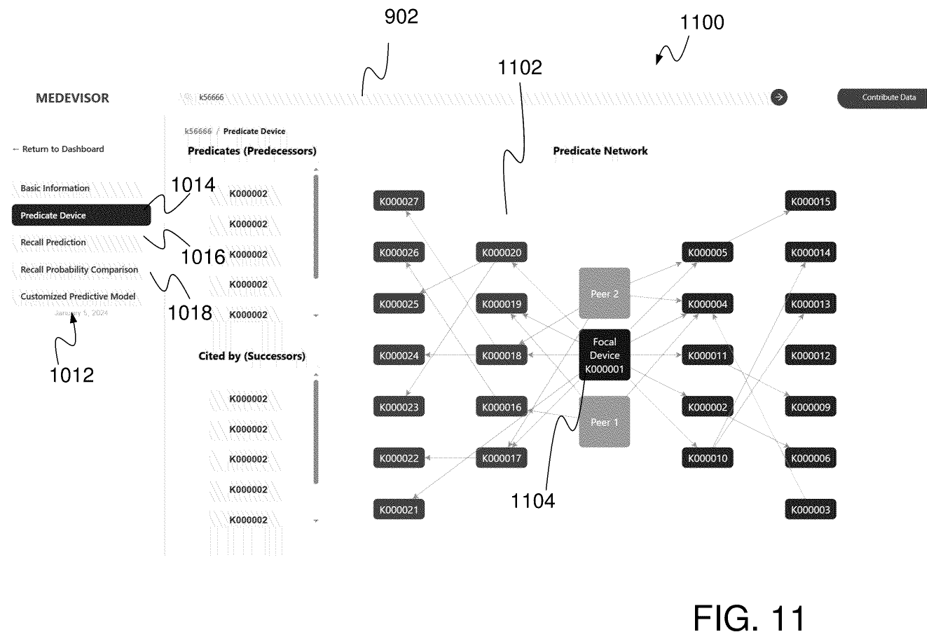

When the user selects tab 1014, server 852 returns predicate devices user interface 1100 of FIG. 11. User interface 1100 includes a predicate devices graph 1102 with the selected device as focal device 1104. Predicate devices graph 1102 is formed as part of feature construction 103 but may also be generated apart from forming features for predictive models 103.

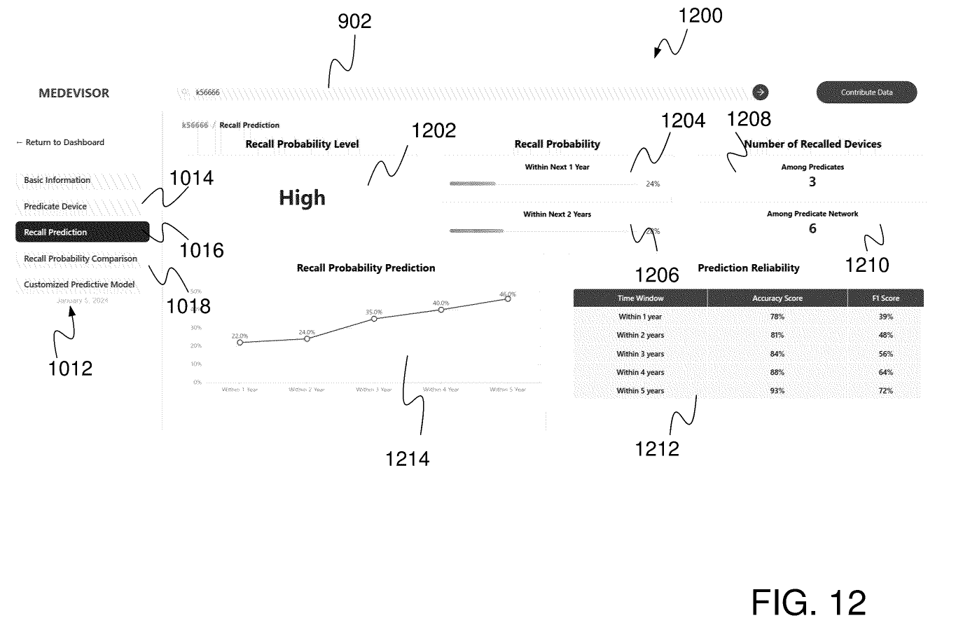

When the user selects tab 1016, server 852 returns a recall prediction user interface 1200 of FIG. 12. Recall prediction user interface 1200 includes a generalized recall risk level 1202, recall probability within a 1-year window 1204, recall probability within a 2-year window 1206, number of recalled products among predecessor predicates 1208, number of recalled products among the predicate network 1210 (predecessors and successors), predictability reliability 1212 and a recall probability graph 1214 showing the recall probability across five time windows.

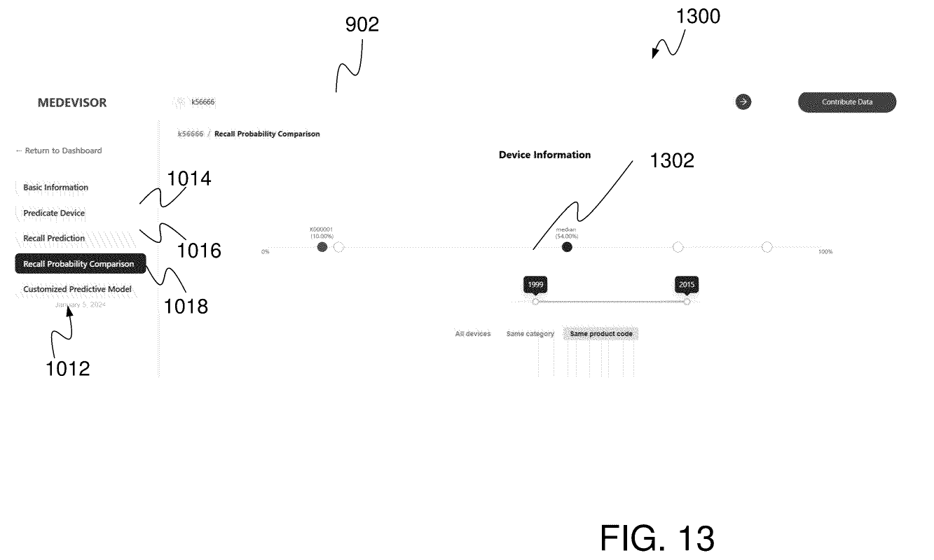

When the user selects tab 1018, server 852 returns recall probability comparison user interface 1300 of FIG. 13. User interface 1300 provides a graph 1302 showing how the recall probability of the selected device compares to the media recall probability of devices in various groupings.

In some embodiments, any suitable computer-readable media can be used for storing instructions for performing the functions and/or processes described herein. For example, in some embodiments, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

The term “article of manufacture” as used herein is intended to encompass a computer program accessible from any computer-readable device, carrier (e.g., non-transitory signals), or media (e.g., non-transitory media). For example, computer-readable media can include but are not limited to magnetic storage devices (e.g., hard disk, floppy disk, magnetic strips, and so on), optical disks (e.g., compact disk (“CD”), digital versatile disk (“DVD′”), and so on), smart cards, and flash memory devices (e.g., card, stick, and so on). Additionally, it should be appreciated that a carrier wave can be employed to carry computer-readable electronic data such as those used in transmitting and receiving electronic mail or in accessing a network such as the Internet or a local area network (“LAN”). Those skilled in the art will recognize that many modifications may be made to these configurations without departing from the scope or spirit of the claimed subject matter.

Certain operations of methods according to the disclosure, or of systems executing those methods, may be represented schematically in the figures or otherwise discussed herein. Unless otherwise specified or limited, representation in the figures of particular operations in particular spatial order may not necessarily require those operations to be executed in a particular sequence corresponding to the particular spatial order. Correspondingly, certain operations represented in the figures, or otherwise disclosed herein, can be executed in different orders than are expressly illustrated or described, as appropriate for particular embodiments of the disclosure. Further, in some embodiments, certain operations can be executed in parallel, including by dedicated parallel processing devices, or separate computing devices configured to interoperate as part of a large system.

As used herein in the context of computer implementation, unless otherwise specified or limited, the terms “component,” “system,” “module,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

The present disclosure has described one or more preferred embodiments, and it should be appreciated that many equivalents, alternatives, variations, and modifications, aside from those expressly stated, are possible and within the scope of the invention.

What is claimed is:

1. A system for estimating a recall probability for a medical device, comprising:
a predicate device database having stored thereon relationships between a plurality of medical devices;
a processor in communication with the predicate device database and configured to:
generate a network of medical devices having a relationship to a focal medical device using the predicate device database;
using the generated network to form features; and
applying the features to a predictive model to determine the recall probability.

2. The system of claim 1 wherein each relationship between two medical devices in the predicate device database is a predicate relationship wherein one of the two medical devices has been listed as a predicate of another of the two medical devices.

3. The system of claim 1 wherein using the generated network to form features further comprises retrieving and using recall data for medical devices in the generated network to form the features.

4. The system of claim 1 wherein applying the features to a predictive model comprises applying the features to at least one Graph Convolution Network.

5. The system of claim 4 wherein applying the features to at least one Graph Convolution Network comprises applying the features to a plurality of Graph Convolution Networks, wherein at least one of the Graph Convolution Networks is constructed for a 1-hop network and one of the Graph Convolution Networks is constructed for a 2-hop network.

6. The system of claim 5 wherein a first Graph Convolution Network is for a 1-hop network at a first time point and a second Graph Convolution Network is for the 1-hop network and a second time point.

7. The system of claim 6 wherein the first Graph Convolution Network and the Second Convolution Network provide respective outputs to a Gated Recurrent Unit.

8. A method for estimating a recall probability for a focal medical device, comprising:
(a) generating a predicate device network for a focal medical device using a computer system;
(b) using the predicate device network to generate features for the focal medical device using a computer system;
(c) applying the features to a predictive model with the computer system, wherein the predictive model has been trained on training data to estimate a medical device recall probability from features associated with a predicate device network; and
(c) outputting a probability that the focal medical device will be recalled within a time window generated by the predictive model

9. The method of claim 8 further comprising applying the features to a second predictive model associated with a second time window and outputting a probability that the focal medical device will be recalled within the second time window generated by the second predictive model.

10. The method of claim 9 wherein predictive model comprises a plurality of branches, each branch being associated with a different number of hops in the predicate device network.

11. The method of claim 10 wherein each branch of the predictive model comprises a Graph Convolution Network trained for the number of hops associated with the branch.

12. The method of claim 9 wherein the predictive model comprises a first plurality of branches receiving temporal features and a second plurality of branches receiving static features, wherein each of the branches of the first plurality of branches is associated with a different number of hops in the predicate network.

13. The method of claim 12 wherein each branch of the first plurality of branches comprises a plurality of Graph Convolution Networks, wherein each Graph Convolution Network along a branch is associated with a separate time point.

14. The method of claim 13 wherein the predictive model further comprises a sequence processing model that receives the outputs of the Graph Convolution Networks at the separate time points.

15. The method of claim 8 wherein using the predicate device network to generate features for the focal medical device further comprises using recall data for medical devices in the predicate device network to generate the features.

16. A method comprising:
applying features to a Graph Convolution Network having an adjacency matrix that is determined from a predicate device network for a focal medical device; and
using the output of the Graph Convolution Network to determine a probability of the focal medical device being recalled.

17. The method of claim 16 wherein the adjacency matrix is determined from the predicate device network by forming a 1-hop network consisting of only medical devices that are one hop away from the focal medical device in the predicate device network.

18. The method claim 16 wherein the adjacency matrix is determined from the predicate device network by forming a 2-hop network consisting of only medical devices that are two hops away from the focal medical device in the predicate device network.

19. The method of claim 16 wherein applying the features to a Graph Convolution Network comprises applying the features to a first Graph Convolution Network and a second Graph Convolution Network, wherein an adjacency matrix for the first Graph Convolution Network is determined from the predicate device network by forming a 1-hop network consisting of only medical devices that are one hop away from the focal medical device in the predicate device network and wherein an adjacency matrix of the second Graph Convolution Network is determined from the predicate device network by forming a 2-hop network consisting of only medical devices that are two hops away from the focal medical device in the predicate device network.

20. The method of claim 16 further comprising:
applying features to a second Graph Convolution Network having an adjacency matrix that is determined from the predicate device network for a focal medical device, wherein the second Graph Convolution Network is associated with a different time point than the Graph Convolution Network; and
using the output of the second Graph Convolution Network to determine the probability of the focal medical device being recalled.
